(19) United States
(12) Patent Application Publication
(10) Pub. No.: US 2020/0061318 A1
(43) Pub. Date: Feb. 27, 2020

(54) AUTOMATIC POSITIVE PRESSURE BREATHING APPARATUS

(71) Applicant: Acare Technology Co., Ltd., New Taipei City (TW)

(72) Inventor: Lin-Ta CHENG, Kaohsiung City (TW)

(21) Appl. No.: 16/233,865

(22) Filed: Dec. 27, 2018

(30) Foreign Application Priority Data
Aug. 21, 2018 (TW) 107211442

(51) Int. Cl. A61M 16/00; A61M 16/10; A61M 16/12

(52) U.S. Cl. CPC A61M 16/024; A61M 16/105; A61M 16/109; A61M 16/12; A61M 2205/3368; A61M 2205/502; A61M 2016/0033; A61M 2016/0027; A61M 2205/52; A61M 16/0003

(57) ABSTRACT

An automatic positive pressure breathing apparatus is provided, applicable to connect an upper respiratory tract of a user, which includes a gas generator, a gas flow sensing device, a humidification device, a heating device, a breathing tube, and a mask device. The gas generator includes a signal processing module and a filter. Wherein the signal processing module includes a control circuit and the filter is embedded in the gas generator. The first connecting end of the gas flow sensing device is positioned at one end of the gas flow sensing device and is connected to one side of the gas generator. The second connecting end is positioned at the other end of the gas flow sensing device. The gas flow sensing device includes a first pressure sensing element, a second pressure sensing element, and a radial structure.

### CROSS-REFERENCE TO RELATED APPLICATION

This application claims priority from Taiwan Patent Application No. 107211442, filed on Aug. 21, 2018, in the Taiwan Intellectual Property Office, the content of which is hereby incorporated by reference in its entirety for all purposes.

### FIELD OF THE INVENTION

The present disclosure relates to an automatic positive pressure breathing apparatus, more particularly to a device equipped with functions of sensing gas flow of supplied gas and rectifying currents. The present disclosure may also relate to an automatic positive pressure breathing apparatus which adjusts supplied gas pressure and adjusts supplied gas frequency on the basis of a user's feedback on gas pressure.

### BACKGROUND OF THE INVENTION

Modern gas flow channels for gas transmission typically only provide conventional pipelines for automatic positive pressure breathing apparatus. In the case where an automatic positive pressure breathing apparatus does not have a stable process of generating gas, the breathable gas passing through the pipeline may not steadily be transmitted to the user's respiratory tract, leading to a loss of gas pressure and increasing turbulent flow.

When the gas pressure provided by the automatic positive pressure breathing apparatus is not the originally set pressure and the gas flow not in a stable condition, the user's breathing may be affected. This would further affect the user's feedback on breathing conditions to the automatic positive pressure breathing apparatus. In this way, it can be shown that the automatic positive pressure breathing apparatus cannot effectively determine the current state of the user's breathing and perform an effective respiratory treatment by itself.

Accordingly, the inventor of the present disclosure has made improvements on apparatus with an inability to steadily supply gas and promptly adjust the respiratory treatment according to the user's feedback on breathing conditions to solve the problems that have been shown for conventional automatic positive pressure breathing apparatus.

### SUMMARY OF THE INVENTION

In view of the aforementioned problems, the present disclosure provides an automatic positive pressure breathing apparatus. When the gas generator generates a first gas, the first gas is imported into the humidification device via the gas flow sensing device. The inside of the automatic positive pressure breathing device includes a radial structure that can effectively reduce the turbulent flow when the first gas is imported into the humidification device. In addition, the gas flow sensing device includes a pressure sensing element that can effectively sense the first gas flow passing through the gas flow sensing device and further have the gas generator adjust the supplied pressure of the first gas. The first gas in the humidification device is imported into the mask device via the breathing tube and is further imported into the user's respiratory tract. The heating device is effectively driven by the control circuit in such a way that the first gas in the humidification device including the water body is attached with vapors. When the automatic positive pressure breathing apparatus is activated, the user generates a second gas and transmits the second gas to the gas generator. The pressure sensing element can be used to effectively determine the user's breathing conditions. Then, the control circuit can be used to adjust the gas pressure supplied by the gas generator and the supplied duration.

On the basis of the aforementioned purpose, the present disclosure provides an automatic positive pressure breathing apparatus, applicable to connect an upper respiratory tract of a user. The automatic positive pressure breathing apparatus includes a gas generator, a gas flow sensing device, a humidification device, a heating device, a breathing tube, and a mask device. The gas generator includes a message processing module and a filter. Wherein, the message processing module is disposed in the gas generator. The message processing module includes a display panel, a control element, a control circuit, and an operation element. Wherein, the control circuit is connected to the display panel, the control element, and the operation element, and the filter is embedded to the gas generator. The first connecting end of the gas flow sensing device is positioned at one end of the gas flow sensing device and is connected to one side of the gas generator. The second connecting end is positioned at the other end of the gas flow sensing device. The gas flow sensing device includes a first pressure sensing element, a second pressure sensing element, and a radial structure. The first pressure sensing element and the second pressure sensing element are connected to the control circuit. The first pressure sensing element is closely disposed on the first connecting end of the gas flow sensing device. The second pressure sensing element is closely disposed on the second connecting end of the gas flow sensing device. The radial structure is disposed in the gas flow sensing device and closely disposed on the second connecting end of the gas flow sensing device. The humidification device is connected to the second connecting end of the gas flow sensing device. The heating device includes a first embedding element and a second embedding element. The heating device is embedded on a bottom of the gas generator by the first embedding elements. The heating device is embedded on the bottom of the humidification device by the second embedding elements. One end of the breathing tube is connected to the humidification device opposite to one side of the heating device. One end of the mask device is connected to the other end of the breathing tube, and the other end of the mask device is connected to the upper respiratory tract of the user. When the gas generator generates the first gas, the first gas is imported into the humidification device via the gas flow sensing device, and the first pressure sensing element generates the first pressure sensing message. When the first gas is imported into the humidification device via the gas flow sensing device, the second pressure sensing element generates a second pressure sensing message, and the first gas is imported into the mask device via the breathing tube to provide gas pressure to the upper respiratory tract of the user.

Preferably, the radial structure may be disposed in the gas flow sensing device and closely disposed on the second connecting end of the gas flow sensing device. The first gas generated by the gas generator flows to the humidification device via structural gaps of the radial structure.

Preferably, when the first pressure sensing element transmits the first pressure sensing message to the operation element, and the second pressure sensing element transmits the second pressure sensing message to the operation element, the operation element operates the first pressure sensing message and the second pressure sensing message to generate a first gas flow sensing message.

Preferably, when the operation element generates the first gas flow sensing message, the operation element transmits the first gas flow sensing message to the control circuit, and the control circuit adjusts a first gas flow of the gas generator by the first gas flow sensing message.

Preferably, the gas generator includes a third pressure sensing element, and the third pressure sensing element may be connected to the control circuit. The third pressure sensing element generates a first sample message, and the third pressure sensing element generates a third pressure sensing message according to the first sample message when the automatic positive pressure breathing apparatus is activated, the user generates a second gas, and the second gas is transmitted to the gas generator.

Preferably, the third pressure sensing element generates second sample messages and the third pressure sensing element generates a fourth pressure sensing message according to the second sample messages when the automatic positive pressure breathing apparatus is activated and the third pressure sensing element does not receive the second gas.

Preferably, the gas generator includes a memory card slot. When a memory card is connected to the memory card slot, the memory card stores a first gas flow sensing message generated in a accordance with the first pressure sensing message and the second pressure sensing message, and stores a second gas flow sensing message or a combination thereof generated in a accordance with the third pressure sensing message and the fourth pressure sensing message.

Preferably, when the operation element receives the third pressure sensing message or the fourth pressure sensing message, the operation element generates a second gas flow sensing message, the operation element transmits the second gas flow sensing message to the control circuit, and the control circuit correspondingly adjusts a supplied gas pressure, a supplied gas frequency, or a combination thereof of the first gas generated by the gas generator via the second gas flow sensing message.

Preferably, a plug is disposed on the bottom of the gas generator. The heating device includes a socket. One end of the plug is connected to the control circuit, and the other end of the plug is connected to the socket. The bottom of the gas generator is attached to the heating device. The heating device includes a heating zone corresponding to one side of the gas generator. The heating zone corresponds to the humidification device connected to the heating device, and the humidification device is attached to the heating zone.

Preferably, when the control elements are triggered to generate a heating message, the control circuit drives the heating zone by the heating message, and the heating zone generates a set temperature. When the humidification device includes a water body, the humidification device generates a moisture zone. When the first gas is transmitted to the moisture zone, the first gas is attached with a plurality of vapors.

In sum, the present disclosure, the automatic positive pressure breathing apparatus, has the following advantages:

(1) Compared with the conventional automatic positive pressure breathing apparatuses, the automatic positive pressure breathing apparatus includes a gas flow sensing device, and the inside of the gas flow sensing device includes a radial structure. When the gas generator generates the first gas, and the first gas flows to the humidification device via the gaps of the radial structure, the turbulent flow of the first gas can effectively be reduced to further make the first gas steadily transmitted to the upper respiratory tract of the user and reduce the loss of the first gas pressure.
(2) Because of the stable gas flow of the first gas generated by the automatic positive pressure breathing apparatus, the gas pressure can effectively be provided for the user to further steadily provide corresponding respiratory treatments according to the user's current breathing conditions. Moreover, through the use of the heating device and the humidification device, the first gas passing through the humidification device can effectively be attached with vapors to prevent the dryness and discomfort of the upper respiratory tract in the user.

To make the aforementioned purposes, technical features, and gains after actual implementation more obvious and understandable, the following description shall be explained in more detail with reference to preferred embodiments together with related figures.

### BRIEF DESCRIPTION OF THE DRAWINGS

FIG. 1 is a schematic diagram of element configuration according to the automatic positive pressure breathing apparatus in the present disclosure.

FIG. 2 is a schematic diagram of actual implementation according to the automatic positive pressure breathing apparatus in the present disclosure.

FIG. 3 is a stereogram of the gas flow sensing device according to the automatic positive pressure breathing apparatus in the present disclosure.

FIG. 4 is a front-view diagram of the gas flow sensing device according to the automatic positive pressure breathing apparatus in the present disclosure.

FIG. 5 is a flow chart of the respiratory treatment according to the automatic positive pressure breathing apparatus in the present disclosure.

### DETAILED DESCRIPTION OF THE ILLUSTRATED EMBODIMENTS

In the present disclosure, it should be noted that the orientation or positional relationship of the terms “up,” “down,” “in,” and the like is based on that shown in the figures, which is only for ease of describing the present disclosure and simplifying the description. The description does not indicate or imply that the referred devices or elements must have a particular orientation, construction, and operation. Therefore, it should not be understood as a restriction on the present disclosure. In addition, the terms “first,” “second,” “third,” and “fourth” are only used for describing purposes and are not to be construed as indicating or implying relative importance.

In the present disclosure, it should be noted that the terms “connect,” “embed,” “dispose,” “import,” “transmit,” “sense,” and “attach” should be considered to be general understanding unless there is a specific regulation or restriction. The specific meanings of the aforementioned terms in the present disclosure shall specifically be understood by a person skilled in the art.

The embodiments of the automatic positive pressure breathing apparatus in the present disclosure are explained with reference to the related figures. For ease of understanding, the same elements in the following embodiments are explained in accordance with the same symbols.

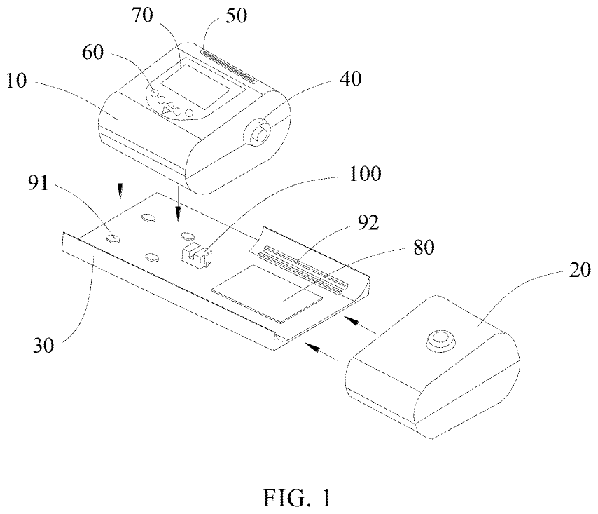

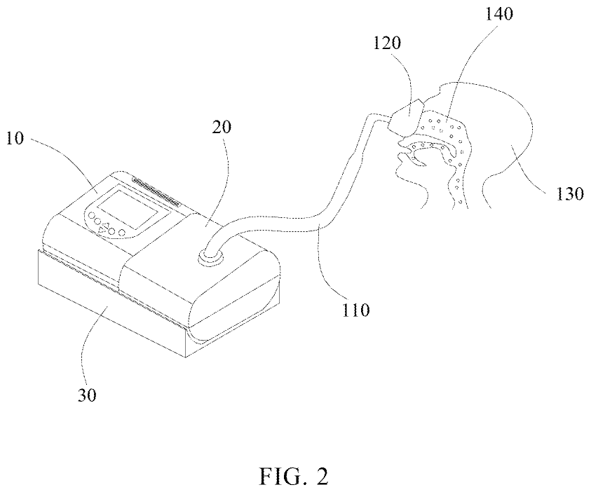

Please refer to FIG. 1 and FIG. 2, respectively illustrating the schematic diagram of element configuration according to the automatic positive pressure breathing apparatus and the schematic diagram of actual implementation according to the automatic positive pressure breathing apparatus in the present disclosure. As shown, the automatic positive pressure breathing apparatus in the present disclosure applies to connecting the upper respiratory tract 140 of the user 130. The automatic positive pressure breathing apparatus includes a gas generator 10, a gas flow sensing device 40, a humidification device 20, a heating device 30, a breathing tube 110, and a mask device 120. The gas generator 10 includes a message processing module and a filter 50. Wherein, the message processing module is disposed in the gas generator 10. The message processing module includes a display panel 70, a control element 60, a control circuit, and an operation element. Wherein, the control circuit is connected to the display panel 70, the control element 60, and the operation element, and the filter 50 is embedded to the gas generator 10. The first connecting end 151 of the gas flow sensing device 40 is positioned at one end of the gas flow sensing device 40 and is connected to one side of the gas generator 10. The second connecting end 152 is positioned at the other end of the gas flow sensing device 40. The gas flow sensing device 40 includes a first pressure sensing element, a second pressure sensing element, and a radial structure 160. The first pressure sensing element and the second pressure sensing element are connected to the control circuit. The first pressure sensing element is closely disposed on the first connecting end 151 of the gas flow sensing device 40. The second pressure sensing element is closely disposed on the second connecting end 152 of the gas flow sensing device 40. The radial structure 160 is disposed in the gas flow sensing device 40 and closely disposed on the second connecting end 152 of the gas flow sensing device 40. The humidification device 20 is connected to the second connecting end 152 of the gas flow sensing device 40. The heating device 30 includes a first embedding element 91 and a second embedding element 92. The heating device 30 is embedded on a bottom of the gas generator 10 by the first embedding elements 91. The heating device 30 is embedded on the bottom of the humidification device 20 by the second embedding elements 92. One end of the breathing tube 110 is connected to the humidification device 20 opposite to one side of the heating device 30. One end of the mask device 120 is connected to the other end of the breathing tube 110, and the other end of the mask device 120 is connected to the upper respiratory tract 140 of the user 130. Wherein, the direction indicated by the arrow in FIG. 1 is the direction in which the gas generator 10 is embedded to the heating device 30 and the humidification device 20 is embedded to the heating device 30.

When the gas generator 10 generates the first gas, the first gas is imported into the humidification device 20 via the gas flow sensing device 40, and the first pressure sensing element generates the first pressure sensing message. When the first gas is imported into the humidification device 20 via the gas flow sensing device 40, the second pressure sensing element generates a second pressure sensing message, and the first gas is imported into the mask device 120 via the breathing tube 110 to provide gas pressure to the upper respiratory tract 140 of the user 130. Wherein, the first pressure sensing message includes a gas pressure value before the first gas is imported into the humidification device 20 via the gas flow sensing device 40, and the second pressure sensing message includes a gas pressure value after the first gas is imported into the humidification device 20 via the gas flow sensing device 40.

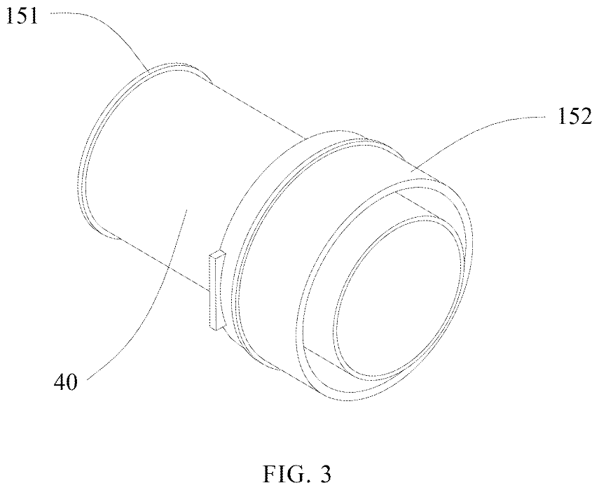

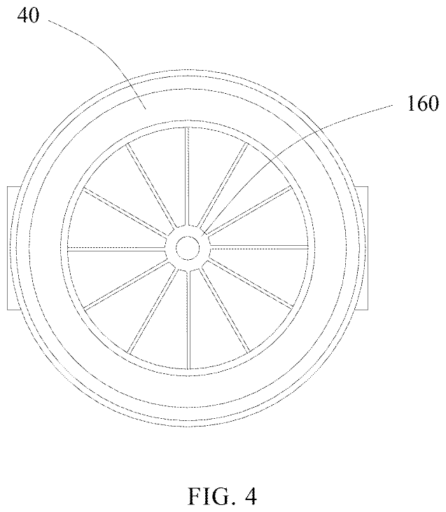

Please refer to FIG. 3 and FIG. 4, respectively illustrating the stereogram of the gas flow sensing device 40 according to the automatic positive pressure breathing apparatus and the front-view diagram of the gas flow sensing device 40 according to the automatic positive pressure breathing apparatus in the present disclosure. As shown, the radial structure 160 may be disposed in the gas flow sensing device 40 and closely disposed on the second connecting end of the gas flow sensing device 40. The first gas generated by the gas generator 10 flows to the humidification device 20 via structural gaps of the radial structure 160. By the structural gaps of the radial structure 160, the turbulent flow possibly generated by the transmission of the first gas to the humidification device 20 may effectively be prevented. The aforementioned descriptions are only explained as examples and not restricted thereto.

Please refer to FIG. 1 and FIG. 2, respectively illustrating the schematic diagram of element configuration according to the automatic positive pressure breathing apparatus and the schematic diagram of actual implementation according to the automatic positive pressure breathing apparatus in the present disclosure. As shown, when the first pressure sensing element transmits the first pressure sensing message to the operation element, and the second pressure sensing element transmits the second pressure sensing message to the operation element, the operation element operates the first pressure sensing message and the second pressure sensing message to generate a first gas flow sensing message. The first gas flow sensing message includes a pressure difference between the gas pressure before the first gas is imported into the humidification device 20 and the gas pressure after the first gas is imported into the humidification device 20 via the gas flow sensing device 40. The aforementioned descriptions are only explained as examples and not restricted thereto.

When the operation element generates the first gas flow sensing message, the operation element transmits the first gas flow sensing message to the control circuit, and the control circuit adjusts a first gas flow of the gas generator 10 by the first gas flow sensing message. The gas flow stability of the first gas imported into the humidifying device 20 via the gas flow sensing device 40 can effectively be controlled. The aforementioned descriptions are only explained as examples and not restricted thereto.

The gas generator 10 includes a third pressure sensing element, and the third pressure sensing element may be connected to the control circuit. The third pressure sensing element generates a first sample message, and the third pressure sensing element generates a third pressure sensing message according to the first sample message when the automatic positive pressure breathing apparatus is activated, the user 130 generates a second gas, and the second gas is transmitted to the gas generator 10.

The third pressure sensing element generates second sample messages and the third pressure sensing element generates a fourth pressure sensing message according to the second sample messages when the automatic positive pressure breathing apparatus is activated and the third pressure sensing element does not receive the second gas.

When the operation element receives the third pressure sensing message or the fourth pressure sensing message, the operation element generates a second gas flow sensing message, the operation element transmits the second gas flow sensing message to the control circuit, the control circuit records the second gas flow sensing message, and the control circuit correspondingly adjusts a supplied gas pressure, a supplied gas frequency, or a combination thereof of the first gas generated by the gas generator 10 via the second gas flow sensing message for a proper respiratory treatment. In addition, when the automatic positive pressure breathing apparatus performs the next respiratory treatment, and the operation element generates a second gas flow sensing message and transmits a second gas flow sensing message to the control circuit, the control circuit correspondingly corrects the second gas flow sensing message of the current treatment according to the second gas flow sensing message recorded in the last respiratory treatment. Therefore, the supplied gas pressure, the supplied gas frequency, or a combination thereof of the first gas generated by the gas generator may further correspondingly be adjusted according to the corrected second gas flow sensing message. In doing so, it is beneficial to optimize the therapeutic effect of the respiratory treatment on the user's breathing conditions. Wherein, the second gas flow sensing message includes the user's 130 breathing conditions. The breathing conditions include Normal, Hypopnea, Flow Limitation, and Apnea. Each breathing condition respectively corresponds to the different supplied gas pressure, supplied gas frequency, or a combination thereof of the first gas. In addition, the criterion for determining the breathing conditions mainly comes from the first sample message and the second sample message respectively generated by the third pressure sensing element in each sleep stage. The first sample message and the second sample message respectively include the second gas pressure, the second gas frequency, or a combination thereof in each sleep stage. The aforementioned descriptions are only explained as examples and not restricted thereto.

A plug is disposed on the bottom of the gas generator 10. The heating device 30 includes a socket 100. One end of the plug is connected to the control circuit, and the other end of the plug is connected to the socket 100. The bottom of the gas generator 10 is attached to the heating device 30. In addition, the heating device 30 includes a heating zone 80 corresponding to one side of the gas generator 10. The heating zone 80 corresponds to the humidification device 20 connected to the heating device 30, and the humidification device 20 is attached to the heating zone 80. The heating device 30 is embedded on the bottom of the gas generator 10 by the first embedding elements 91. The heating device 30 is embedded on the bottom of the humidification device 20 by the second embedding elements 92.

When the control elements 60 are triggered to generate a heating message, the control circuit drives the heating zone 80 by the heating message, and the heating zone 80 generates a set temperature. When the humidification device 20 includes a water body, the humidification device 20 generates a moisture zone. When the first gas is transmitted to the moisture zone, the first gas is attached with a plurality of vapors.

The gas generator 10 includes a memory card slot. When a memory card is connected to the memory card slot, the memory card stores a first gas flow sensing message generated in a accordance with the first pressure sensing message and the second pressure sensing message, and stores a second gas flow sensing message or a combination thereof generated in a accordance with the third pressure sensing message and the fourth pressure sensing message.

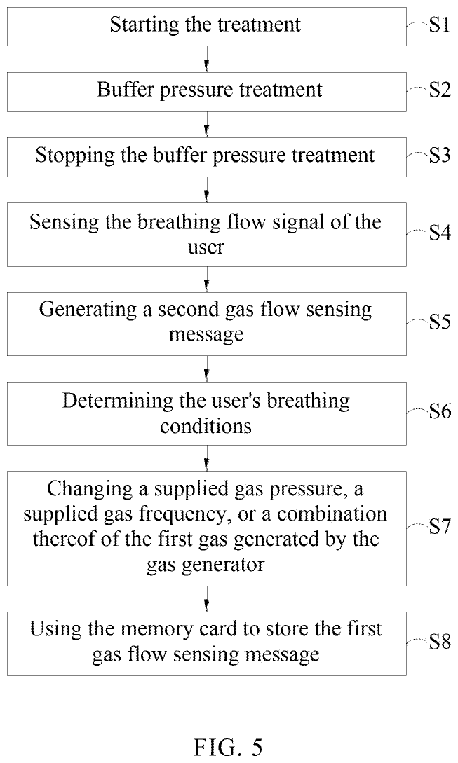

Please refer to FIG. 5, illustrating the flow chart of the respiratory treatment according to the automatic positive pressure breathing apparatus in the present disclosure. As shown, when the automatic positive pressure breathing apparatus is activated and triggers the control element 60, the steps S1, S2, S3, S4, S5, S6, S7, and S8 may sequentially proceed. Wherein, S1 is to start the treatment, and S2 is to generate the set first gas by the gas generator 10. Moreover, the first gas is transmitted to the upper respiratory tract 140 of the user 130 via the gas flow sensing device 40, the humidification device 20, the breathing tube 110, and the mask device 120 to provide the user 130 with a buffer pressure treatment. Step S3 is to stop the buffer pressure treatment. That is, the pressure value of the first gas is adjusted to be higher gradually by the gas generator 10. When the pressure value of the first gas can effectively help adjust the user's 130 breathing conditions, the gas generator 10 stops increasing the pressure value of the first gas. Step S4 is to sense the breathing flow signal of the user 130. That is, when the second gas generated by the user 130 is transmitted to the third pressure sensing element of the gas generating device, the third pressure sensing element generates a first sample message, and the third pressure sensing element generates the third pressure sensing message according to the first sample message. When the user 130 does not generate the second gas, the third pressure sensing element generates the second sample message, and the third pressure sensing element generates the fourth pressure sensing message according to the second sample message. Step S5 is that when the third pressure sensing element transmits the third pressure sensing message or the fourth pressure sensing message to the operation element, the operation element may generate a second gas flow sensing message. Step S6 is that when the operation element transmits the second gas flow sensing message to the control circuit, the control circuit determines the user's 130 breathing conditions by the second gas flow sensing message. Step S7 is that the control circuit correspondingly adjusts a supplied gas pressure, a supplied gas frequency, or a combination thereof of the first gas generated by the gas generator 10 by the breathing conditions corresponding to the second gas flow sensing message. Step S8 is that the memory card connected to the memory card slot stores the first gas flow sensing message, the second gas flow sensing message, and a combination thereof. The aforementioned descriptions are only explained as examples and not restricted thereto.

For instance, when the user 130 activates the automatic positive pressure breathing apparatus, the gas generator 10 generates the first gas which is further imported into the humidification device 20 via gas flow sensing device 40. The first gas in the humidification device 20 is transmitted to the mask device 120 via the breathing tube 110 to further import the first gas into the upper respiratory tract 140 of the user 130. Wherein, when the first gas is imported into the humidification device 20 via the gas flow sensing device 40, the first pressure sensing element generates the first pressure sensing message. When the first gas is imported into the humidification device 20 via the gas flow sensing device 40, the second pressure sensing element generates the second pressure sensing message. When the operation element receives the first pressure sensing message and the second pressure sensing message, the operation element operates the first pressure sensing message and the second pressure sensing message to generate a first gas flow sensing message. The first gas flow sensing message includes a pressure difference between the gas pressure before the first gas is imported into the humidification device 20 and the gas pressure after the first gas is imported into the humidification device 20 via the gas flow sensing device 40. The aforementioned descriptions are only explained as examples and not restricted thereto.

For instance, the radial structure 160 may be disposed in the gas flow sensing device 40 and closely disposed on the second connecting end 152 of the gas flow sensing device 40. The first gas generated by the gas generator 10 is transmitted to the humidification device 20 via structural gaps of the radial structure 160. By the structural gaps of the radial structure 160, the turbulent flow possibly generated by the transmission of the first gas to the humidification device 20 may effectively be prevented. The pressure difference between the gas pressure before the first gas is imported into the humidification device 20 and after the first gas is imported into the humidification device 20 via the gas flow sensing device 40 can also be effectively reduced. The aforementioned descriptions are only explained as examples and not restricted thereto.

For instance, when the user 130 activates the automatic positive pressure breathing apparatus, and the second gas generated by the user 130 is transmitted to the third pressure sensing element of the gas generating device, the third pressure sensing element generates a first sample message, and the third pressure sensing element generates the third pressure sensing message according to the first sample message. When the user 130 does not generate the second gas, the third pressure sensing element generates the second sample message, and the third pressure sensing element generates the fourth pressure sensing message according to the second sample message. In this way, the third pressure sensing message and the fourth pressure sensing message can accurately reflect user's breathing conditions. When the operation element receives the third pressure sensing message and the fourth pressure sensing message, the operation element generates second gas flow sensing message. When the operation element transmits the second gas flow sensing message to the control circuit, the control circuit records the second gas flow sensing message, and the control circuit correspondingly adjusts a supplied gas pressure, a supplied gas frequency, or a combination thereof of the first gas generated by the gas generator 10 via the second gas flow sensing message for a proper respiratory treatment. When the automatic positive pressure breathing apparatus performs the next respiratory treatment, and the operation element generates a second gas flow sensing message and transmits a second gas flow sensing message to the control circuit, the control circuit correspondingly corrects the second gas flow sensing message of the current treatment according to the second gas flow sensing message recorded in the last respiratory treatment. Therefore, the supplied gas pressure, the supplied gas frequency, or a combination thereof of the first gas generated by the gas generator may further correspondingly be adjusted according to the corrected second gas flow sensing message. In doing so, it is beneficial to optimize the therapeutic effect of the respiratory treatment on the user's breathing conditions. Wherein, the second gas flow sensing message includes the user's 130 breathing conditions. The breathing conditions include Normal, Hypopnea, Flow Limitation, and Apnea. Each breathing condition respectively corresponds to the different supplied gas pressure, supplied gas frequency, or a combination thereof of the first gas. In addition, the criterion for determining the breathing conditions mainly comes from the first sample message and the second sample message respectively generated by the third pressure sensing element in each sleep stage. The first sample message and the second sample message respectively include the second gas pressure, the second gas frequency, or a combination thereof in each sleep stage. The aforementioned descriptions are only explained as examples and not restricted thereto.

Compared with the conventional automatic positive pressure breathing apparatuses, the automatic positive pressure breathing apparatus includes a gas flow sensing device 40, and the inside of the gas flow sensing device 40 includes a radial structure 160. When the gas generator 10 generates the first gas, and the first gas flows to the humidification device 20 via the gaps of the radial structure 160, the turbulent flow of the first gas can effectively be reduced to further make the first gas steadily transmitted to the upper respiratory tract 140 of the user 130 and reduce the loss of the first gas pressure. Because of the stable gas flow of the first gas generated by the automatic positive pressure breathing apparatus, the gas pressure can effectively be provided for the user 130 to further steadily provide corresponding respiratory treatments according to the user's 130 current breathing conditions. Moreover, through the use of the heating device 30 and the humidification device 20, the first gas passing through the humidification device 130 can effectively be attached with vapors to prevent the dryness and discomfort in the upper respiratory tract 140 of the user 130.

What is stated above is only illustrative examples which do not limit the present disclosure. Any spirit and scope without departing from the present invention as to equivalent modifications or alterations is intended to be included in the following claims.

What is claimed is:

1. An automatic positive pressure breathing apparatus, applicable to connect an upper respiratory tract of a user, and the upper respiratory tract comprising:
a gas generator, comprising:
a message processing module disposed in the gas generator; the message processing module comprising a display panel, a plurality of control elements, a control circuit, and an operation element; wherein, the control circuit is connected to the display panel, the plurality of control elements, and the operation element; and
a filter embedded in the gas generator;
a gas flow sensing device, comprising:
a first connecting end positioned at one end of the gas flow sensing device and connected to one side of the gas generator;
a second connecting end positioned at the other end of the gas flow sensing device;
a first pressure sensing element connected to the control circuit and closely disposed on the first connecting end of the gas flow sensing device;
a second pressure sensing element connected to the control circuit and closely disposed on the second connecting end of the gas flow sensing device;
a radial structure disposed in the gas flow sensing device and closely disposed on the second connecting end of the gas flow sensing device;
a humidification device connected to the second connecting end of the gas flow sensing device;
a heating device comprising a plurality of first embedding elements and a plurality of second embedding element, the heating device embedded on a bottom of the gas generator by the plurality of first embedding elements, and the heating device embedded on the bottom of the humidification device by the plurality of second embedding elements;
a breathing tube, one end thereof connected to the humidification device opposite to one side of the heating device; and
a mask device, one end thereof connected to the other end of the breathing tube, and the other end of the mask device connected to the upper respiratory tract of the user;
wherein, when the gas generator generates a first gas, the first gas is imported into the humidification device via the gas flow sensing device, and the first pressure sensing element generates a first pressure sensing message; when the first gas is imported into the humidification device via the gas flow sensing device, the second pressure sensing element generates a second pressure sensing message, and the first gas is imported into the mask device via the breathing tube to provide an air pressure to the upper respiratory tract of the user.

2. The automatic positive pressure breathing apparatus according to claim 1, wherein the radial structure is disposed in the gas flow sensing device and closely positioned at the second connecting end of the gas flow sensing device, and the first gas generated by the gas generator flows to the humidification device via a plurality of structural gaps of the radial structure.

3. The automatic positive pressure breathing apparatus according to claim 1, wherein when the first pressure sensing element transmits the first pressure sensing message to the operation element, and the second pressure sensing element transmits the second pressure sensing message to the operation element, the operation element operates the first pressure sensing message and the second pressure sensing message to generate a first gas flow sensing message.

4. The automatic positive pressure breathing apparatus according to claim 3, wherein when the operation element generates the first gas flow sensing message, the operation element transmits the first gas flow sensing message to the control circuit, and the control circuit adjusts a first gas flow of the gas generator by the first gas flow sensing message.

5. The automatic positive pressure breathing apparatus according to claim 1, wherein the gas generator comprises a third pressure sensing element, and the third pressure sensing element is connected to the control circuit; The third pressure sensing element generates a plurality of first sample messages and the third pressure sensing element generates a third pressure sensing message according to the plurality of first sample messages when the automatic positive pressure breathing apparatus is booted, the user generates a second gas, and the second gas is transmitted to the gas generator.

6. The automatic positive pressure breathing apparatus according to claim 5, wherein the third pressure sensing element generates a plurality of second sample messages and the third pressure sensing element generates a fourth pressure sensing message according to the plurality of second sample messages when the automatic positive pressure breathing apparatus is booted and the third pressure sensing element does not receive the second gas.

7. The automatic positive pressure breathing apparatus according to claim 6, wherein the gas generator comprises a memory card slot; when a memory card is connected to the memory card slot, the memory card stores a first gas flow sensing message generated in a accordance with the first pressure sensing message and the second pressure sensing message, and stores a second gas flow sensing message or a combination thereof generated in a accordance with the third pressure sensing message and the fourth pressure sensing message.

8. The automatic positive pressure breathing apparatus according to claim 6, wherein when the operation element receives the third pressure sensing message or the fourth pressure sensing message, the operation element generates a second gas flow sensing message, the operation element transmits the second gas flow sensing message to the control circuit, and the control circuit correspondingly adjusts a supplied gas pressure, a supplied gas frequency, or a combination thereof of the first gas generated by the gas generator.

9. The automatic positive pressure breathing apparatus according to claim 1, wherein a plug is disposed on the bottom of the gas generator, the heating device comprises a socket, one end of the plug is connected to the control circuit, and the other end of the plug is connected to the socket; the bottom of the gas generator is attached to the heating device, the heating device comprises a heating zone corresponding to one side of the gas generator, the heating zone corresponds to the humidification device connected to the heating device, and the humidification device is attached to the heating zone.

10. The automatic positive pressure breathing apparatus according to claim 9, wherein when the plurality of control elements are triggered to generate a heating message, the control circuit drives the heating zone by the heating message, the heating zone generates a set temperature; when the humidification device comprises a water body, the humidification device generates a moisture zone; when the first gas is transmitted to the moisture zone, the first gas is attached to a plurality of vapors.
